(19) United States
(12) Patent Application Publication
(10) Pub. No.: US 2003/0003500 A1
(43) Pub. Date: Jan. 2, 2003

(54) Capillary array-based sample screening

(75) Inventor: William Michael Lafferty, Encinitas, CA (US)

Correspondence Address: Lisa A. Haile, Ph.D., Gray Cary Ware & Freidenrich LLP, Suite 1100, 4365 Executive Drive, San Diego, CA, 92121-2133, US

(73) Assignee: Diversa Coporation

(21) Appl. No.: 10/232,869

(22) Filed: Aug. 30, 2002

Related U.S. Application Data
(62) Division of application No. 09/894,956, filed on Jun. 27, 2001.
Application No. 09/894,956 is a continuation-in-part of application No. 09/790,321, filed on Feb. 21, 2001.
Application No. 09/790,321 is a division of application No. 09/687,219, filed on Oct. 12, 2000.
Application No. 09/687,219 is a continuation-in-part of application No. 09/444,112, filed on Nov. 22, 1999.
Application No. 09/687,219 is a continuation-in-part of application No. 09/636,778, filed on Aug. 11, 2000.
Application No. 09/636,778 is a continuation of application No. 09/098,206, filed on Jun. 16, 1998, now Pat. No. 6,174,673.
Application No. 09/098,206 is a continuation-in-part of application No. 08/876,276, filed on Jun. 16, 1997.

(51) Int. Cl. C12Q 1/68; C12M 1/34

(52) U.S. Cl. 435/6; 435/287.2

(57) ABSTRACT

A sample screening platform, system and method for screening and recovery of detectable samples. The platform includes a capillary array including a plurality of capillaries. Each capillary includes at least one wall defining a lumen for retaining the detectable sample. The detectable sample is introduced to and retained in the lumen by capillary forces. In a method of incubating the sample, a second liquid is introduced into the capillary containing the sample. A detection and recovery system includes an optical system for detecting the sample, and a recovery mechanism adapted to contact a capillary containing the sample. The recovery mechanism is further adapted to recover the sample from the capillary, and expel the recovered sample for analysis.

### CROSS REFERENCE TO RELATED APPLICATIONS

This application is a divisional of U.S. patent application Ser. No. 09/894,956, filed Jun. 27, 2001, which is a continuation-in-part of U.S. patent application Ser. No. 09/790,321, filed Feb. 21, 2001, which is a divisional of U.S. patent application Ser. No. 09/687,219, filed Oct. 12, 2000, which is a continuation-in-part of U.S. patent application Ser. No. 09/636,778, filed Aug. 11, 2000, which is a continuation of U.S. patent application Ser. No. 09/098,206, filed Jun. 16, 1998, now U.S. Pat. No. 6,174,673, which is a continuation-in-part of U.S. patent application Ser. No. 08/876,276, filed Jun. 16, 1997; this application also claims priority under 35 U.S.C. §120 as a continuation in part of which is a continuation-in-part of U.S. patent application Ser. No. 09/790,321, filed Feb. 21, 2001, which is a divisional of U.S. patent application Ser. No. 09/687,219, filed Oct. 12, 2000, which is a continuation-in-part of U.S. patent application Ser. No. 09/444,112, filed Nov. 22, 1999, which is a continuation-in-part of U.S. patent application Ser. No. 09/098,206, filed Jun. 16, 1998, now U.S. Pat. No. 6,174,673, which is a continuation-in-part of U.S. patent application Ser. No. 08/876,276, filed Jun. 16, 1997, all of the contents of which are incorporated by reference in their entirety herein.

### FIELD OF THE INVENTION

The present invention relates generally to screening and identification of new bioactive molecules. More specifically, the present invention relates to a capillary array platform for screening samples, and methods of the platform's use and manufacture.

Current platforms for screening micro-scale particles of interest include plates that are formed with small wells, or through-holes. The wells or through-holes are used to hold a sample to be analyzed. The sample typically contains the particles of interest. When wells are used, complex and inefficient sample delivery and extraction systems must be used in order to deposit the sample into the wells on the plate, and remove the sample from the wells for further analysis. Wells-based platforms have a bottom, for which gravity is primarily used for suspending the sample on the plate to develop the particulate or incubate cells of interest.

Another type of platform uses through-holes, which are typically machined into a plate by one of a number of well-known methods. Through-holes rely on capillary forces for introducing the sample to the plate, and utilize surface tension for suspending the sample in the through-holes. However, typical through-hole-based devices are limited to relatively small aspect ratios, or the ratio of length to internal diameter of the hole. A small aspect ratio yields greater evaporative loss of a liquid contained in the hole, and such evaporation is difficult to control. Through-holes are also limited in their functionality. For example, the process of forming through-holes in a plate usually does not allow for the use of various materials to line the inside of the holes, or to clad the outside of the holes.

Accordingly, there is a need for an improved sample holding and screening platform.

### SUMMARY OF THE INVENTION

The invention provides a system and method for holding and screening samples. According to one embodiment of the invention, a sample screening apparatus includes a plurality of capillaries formed into an array of adjacent capillaries, wherein each capillary comprises at least one wall defining a lumen for retaining a sample. The apparatus further includes interstitial material disposed between adjacent capillaries in the array, and one or more reference indicia formed within of the interstitial material.

According to another embodiment of the invention, a capillary for screening a sample, wherein the capillary is adapted for being bound in an array of capillaries, includes a first wall defining a lumen for retaining the sample, and a second wall formed of a filtering material, for filtering excitation energy provided to the lumen to excite the sample.

According to yet another embodiment of the invention, a method for incubating a bioactivity or biomolecule of interest includes the steps of introducing a first component into at least a portion of a capillary of a capillary array, wherein each capillary of the capillary array comprises at least one wall defining a lumen for retaining the first component, and introducing an air bubble into the capillary behind the first component. The method further includes the step of introducing a second component into the capillary, wherein the second component is separated from the first component by the air bubble.

In yet another embodiment of the invention, a method of incubating a sample of interest includes introducing a first liquid labeled with a detectable particle into a capillary of a capillary array, wherein each capillary of the capillary array comprises at least one wall defining a lumen for retaining the first liquid and the detectable particle, and wherein the at least one wall is coated with a binding material for binding the detectable particle to the at least one wall. The method further includes removing the first liquid from the capillary tube, wherein the bound detectable particle is maintained within the capillary, and introducing a second liquid into the capillary tube.

Another embodiment of the invention includes a recovery apparatus for a sample screening system, wherein the system includes a plurality of capillaries formed into an array. The recovery apparatus includes a recovery tool adapted to contact at least one capillary of the capillary array and recover a sample from the at least one capillary. The recovery apparatus further includes an ejector, connected with the recovery tool, for ejecting the recovered sample from the recovery tool.

### BRIEF DESCRIPTION OF THE DRAWING

FIG. 1A shows an example of dimensions of a capillary array of the invention.

FIG. 1B illustrates an array of capillary arrays.

FIG. 2 shows a top cross-sectional view of a capillary array.

FIG. 3 is a schematic depicting the excitation of and emission from a within the capillary lumen according to one embodiment of the invention.

FIG. 4 is a schematic depicting the filtering of excitation and emission light to and from a sample within the capillary lumen according to an alternative embodiment of the invention.

FIG. 5 illustrates an embodiment of the invention in which a capillary array is wicked by contacting a sample containing cells, and humidified in a humidified incubator followed by imaging and recovery of cells in the capillary array.

FIG. 6 illustrates a method for incubating a sample in a capillary tube by an evaporative and capillary wicking cycle.

FIG. 7A shows a portion of a surface of a capillary array on which condensation has formed.

FIG. 7B shows the portion of the surface of the capillary array, depicted in FIG. 7A, in which the surface is coated with a hydrophobic layer to inhibit condensation near an end of individual capillaries.

FIGS. 8A-C depict a method of retaining at least two components within a capillary.

FIG. 9A depicts capillary tubes containing paramagnetic beads and cells.

FIG. 9B depicts the use of the paramagnetic beads to stir a sample in a capillary tube.

FIG. 10 depicts an excitation apparatus for a detection system according to an embodiment of the invention.

FIG. 11 illustrates a system for screening samples using a capillary array according to an embodiment of the invention.

FIG. 12A illustrates one example of a recovery technique useful for recovering a sample from a capillary array. In this depiction a needle is contacted with a capillary containing a sample to be obtained. A vacuum is created to evacuate the sample from the capillary tube and onto a filter.

FIG. 12B illustrates one sample recovery method in which the recovery device has an outer diameter greater than the inner diameter of the capillary from which a sample is being recovered.

FIG. 12C illustrates another sample recovery method in which the recovery device has an outer diameter approximately equal to or less than the inner diameter of the capillary.

FIG. 12D shows the further processing of the sample once evacuated from the capillary.

### DETAILED DESCRIPTION OF THE PREFERRED EMBODIMENTS

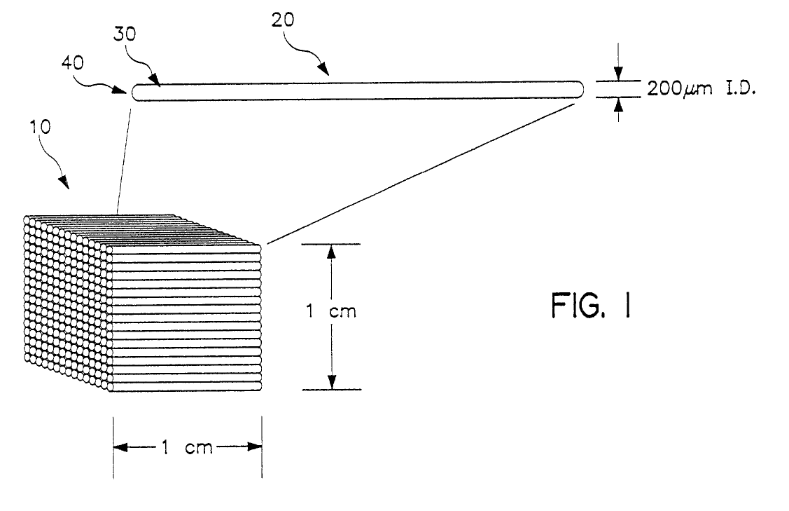

FIG. 1A shows a capillary array (10) which includes a plurality of individual capillaries (20) having at least one outer wall (30) defining a lumen (40). The outer wall (30) of the capillary (20) can be one or more walls fused together. Similarly, the wall can define a lumen (40) that is cylindrical, square, hexagonal or any other geometric shape so long as the walls form a lumen for retention of a liquid or sample. The capillaries (20) of the capillary array (10) are held together in close proximity to form a planar structure. The capillaries (20) can be bound together, by being fused (e.g., where the capillaries are made of glass), glued, bonded, or clamped side-by-side. The capillary array (10) can be formed of any number of individual capillaries (20). In an embodiment, the capillary array includes 100 to 4,000,000 capillaries (20). In one embodiment, the capillary array includes 100 to 500,000,000 capillaries (20). In one embodiment, the capillary array includes 100,000 capillaries (20). In one specific embodiment, the capillary array (10) can be formed to conform to a microtiter plate footprint, i.e. 127.76 mm by 85.47 mm, with tolerances. The capillary array (10) can have a density of 500 to more than 1,000 capillaries (20) per cm², or about 5 capillaries per mm². For example, a microtiter plate size array of 3 um capillaries would have about 500 million capillaries.

The capillaries (20) are preferably formed with an aspect ratio of 50:1. In one embodiment, each capillary (20) has a length of approximately 10 mm, and an internal diameter of the lumen (40) of approximately 200 μm. However, other aspect ratios are possible, and range from 10:1 to well over 1000:1. Accordingly, the thickness of the capillary array can vary from 0.5 mm to over 10 cm. Individual capillaries (20) have an inner diameter that ranges from 3-500 μm and 0-500 μm. A capillary (20) having an internal diameter of 200 μm and a length of 1 cm has a volume of approximately 0.3 μl. The length and width of each capillary (20) is based on a desired volume and other characteristics discussed in more detail below, such as evaporation rate of liquid from within the capillary and the like. Capillaries of the invention may include a volume as low as 250 nanoliters/well.

In accordance with one embodiment of the invention, one or more particles are introduced into each capillary (20) for screening. Suitable particles include cells, cell clones, and other biological matter, chemical beads, or any other particulate matter. The capillaries (20) containing particles of interest can be introduced with various types of substances for causing an activity of interest. The introduced substance can include a liquid having a developer or nutrients, for example, which assists in cell growth and which results in the production of enzymes. Or, a chemical solution containing new particles can cause a combining event with other chemical beads already introduced into one or more capillaries (20). The particles and resulting activity of interest are screened and analyzed using the capillary array (10) according to the present invention. In one embodiment, the activity produces a change in properties of matter within the capillary (20), such as optical properties of the particles. Each capillary can act as a waveguide for guiding detectable light energy or property changes to an analyzer.

The capillaries (20) can be made according to various manufacturing techniques. In one particular embodiment, the capillaries (20) are manufactured using a hollow-drawn technique. A cylindrical, or other hollow shape, piece of glass is drawn out to continually longer lengths according to known techniques. The piece of glass is preferably formed of multiple layers. The drawn glass is then cut into portions of a specific length to form a relatively large capillary. The capillary portions are next bundled into an array of relatively large capillaries, and then drawn again to increasingly narrower diameters. During the drawing process, or when the capillaries are formed to a desired width, application of heat can fuse interstitial areas of adjacent capillaries together.

In an alternative embodiment, a glass etching process is used. Preferably, a solid tube of glass is drawn out to a particular width, cut into portions of a specific length, and drawn again. Then, each solid tube portion is center-etched with an acid or other etchant to form a hollow capillary. The tubes can be bound or fused together before or after the etch process.

A number of capillary arrays (10) can be connected together to form an array of arrays (12), as shown in FIG. 1B. The capillary arrays (10) can be glued together. Alternatively, the capillary arrays (10) can be fused together. According to this technique, the array of arrays (12) can have any desired size or footprint, formed of any number of high-precision capillary arrays (10).

A large number of materials can be suitably used to form a capillary array according to the invention and depending on the manufacturing technique used, including without limitation, glass, metal, semiconductors such as silicon, quartz, ceramics, or various polymers and plastics including, among others, polyethylene, polystyrene, and polypropylene. The internal walls of the capillary array, or portions thereof, may be coated or silanized to modify their surface properties. For example, the hydrophilicity or hydrophobicity may be altered to promote or reduce wicking or capillary action, respectively. The coating material includes, for example, ligands such as avidin, streptavidin, antibodies, antigens, and other molecules having specific binding affinity or which can withstand thermal or chemical sterilization.

While the above-described manufacturing techniques and materials yield high precision micro-sized capillaries and capillary arrays, the size, spacing and alignment of the capillaries within an array may be non-uniform. In some instances, it is desirable to have two capillary arrays make contact in as close alignment as possible, such as, for example, to transfer liquid from capillaries in a first capillary array to capillaries in a second capillary array. One capillary array according to the invention may be cut horizontally along its thickness, and separated to form two capillary arrays. The two resulting capillary arrays will each include at least one surface having capillary openings of substantially identical size, spacing and alignment, and suitable for contacting together for transferring liquid from one resulting capillary array to the other.

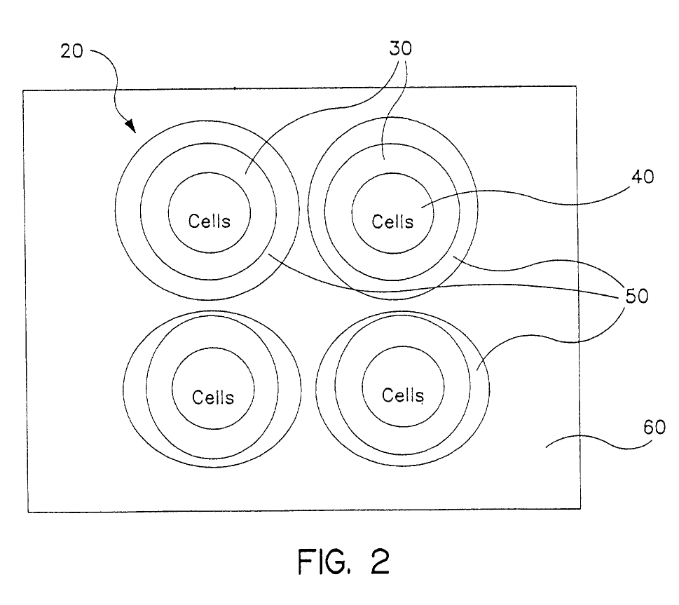

FIG. 2 shows a horizontal cross section of a portion of an array of capillaries (20). Capillary (20) is shown having a first cylindrical wall (30), a lumen (40), a second exterior wall (50), and interstitial material (60) separating the capillary tubes in the array (10). In this embodiment, the cylindrical wall (30) is comprised of a sleeve glass, while exterior wall (50) is comprised of an extra mural absorption (EMA) glass to minimize optical crosstalk among neighboring capillaries (20).

A capillary array may optionally include reference indicia (22) for providing a positional or alignment reference. The reference indicia (22) may be formed of a pad of glass extending from the surface of the capillary array, or embedded in the interstitial material (60). In one embodiment, the reference indicia (22) are provided at one or more comers of a microtiter plate formed by the capillary array. According to the embodiment, a comer of the plate or set of capillaries may be removed, and replaced with the reference indicia (22). The reference indicia (22) may also be formed at spaced intervals along a capillary array, to provide an indication of a subset of capillaries (20).

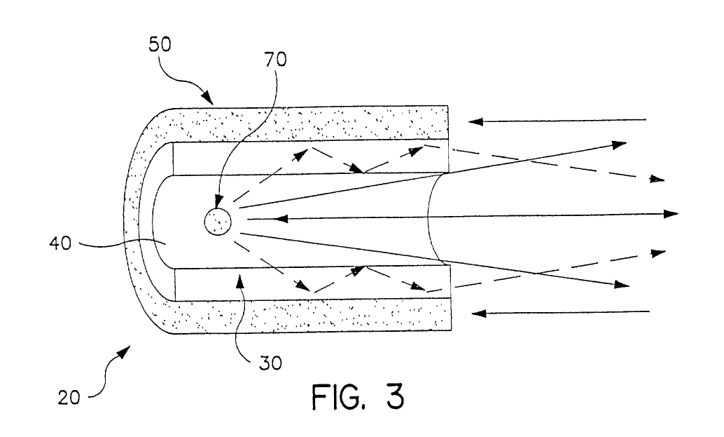

FIG. 3 depicts a vertical cross-section of a capillary of the invention. The capillary (20) includes a first wall (30) defining a lumen (40), and a second wall (50) surrounding the first wall (30). In one embodiment, the second wall (50) has a lower index of refraction than the first wall (30). In one embodiment, the first wall (30) is sleeve glass having a high index of refraction, forming a waveguide in which light from excited fluorophores travels. In the exemplary embodiment, the second wall (50) is black EMA glass, having a low index of refraction, forming a cladding around the first wall (30) against which light is refracted and directed along the first wall (30) for total internal reflection within the capillary (20). The second wall (50) can thus be made with any material that reduces the “cross-talk” or diffusion of light between adjacent capillaries. Alternatively, the inside surface of the first wall (30) can be coated with a reflective substance to form a mirror, or mirror-like structure, for specular reflection within the lumen (40).

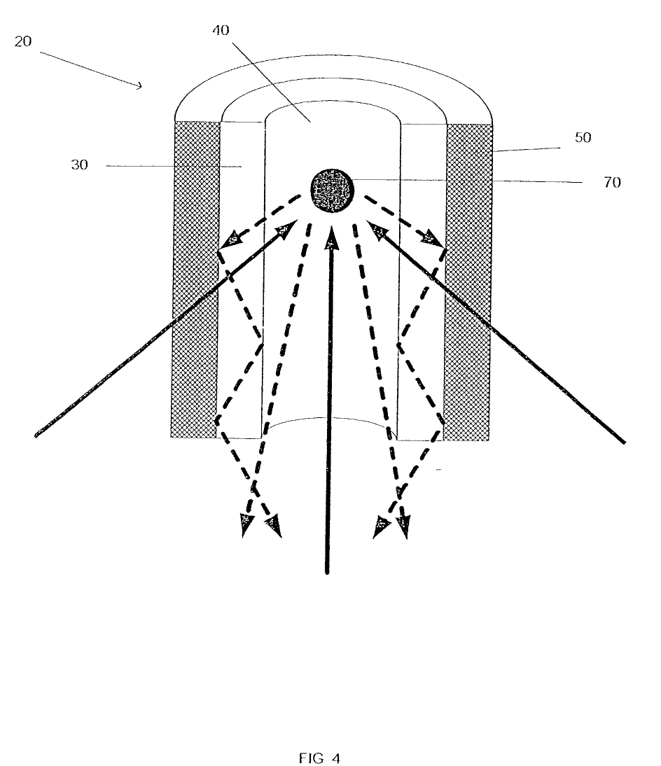

Many different materials can be used in forming the first and second walls, creating different indices of refraction for desired purposes. A filtering material can be formed around the lumen (40) to filter energy to and from the lumen (40) as depicted in FIG. 4. In one embodiment, the inner wall of the first wall (30) of each capillary of the array, or portion of the array, is coated with the filtering material. In another embodiment, the second wall (50) includes the filtering material. For instance, the second wall (50) can be formed of the filtering material, such as filter glass for example, or in one exemplary embodiment, the second wall (50) is EMA glass that is doped with an appropriate amount of filtering material. The filtering material can be formed of a color other than black and tuned for a desired excitation/emission filtering characteristic.

The filtering material allows transmission of excitation energy into the lumen (40), and blocks emission energy from the lumen (40) except through one or more openings at either end of the capillary (20). In FIG. 4, excitation energy is illustrated as a solid line, while emission energy is indicated by a broken line. When the second wall (50) is formed with a filtering material as shown in FIG. 4, certain wavelengths of light representing excitation energy are allowed through to the lumen (40), and other wavelengths of light representing emission energy are blocked from exiting, except as directed within and along the first wall (30). The entire capillary array, or a portion thereof, can be tuned to a specific individual wavelength or group of wavelengths, for filtering different bands of light in an excitation and detection process.

A particle (70) is depicted within the lumen (40). During use, an excitation light is directed into the lumen (40) contacting the particle (70) and exciting a reporter fluorescent material causing emission of light. The emitted light travels the length of the capillary until it reaches a detector. One advantage of an embodiment of the present invention, where the second wall (50) is black EMA glass, is that the emitted light cannot cross contaminate adjacent capillary tubes in a capillary array. In addition, the black EMA glass refracts and directs the emitted light towards either end of the capillary tube thus increasing the signal detected by an optical detector (e.g., a CCD camera and the like).

In a detection process using a capillary array of the invention, an optical detection system is aligned with the array, which is then scanned for one or more bright spots, representing either a fluorescence or luminescence associated with a “positive.” The term “positive” refers to the presence of an activity of interest. Again, the activity can be a chemical event, or a biological event.

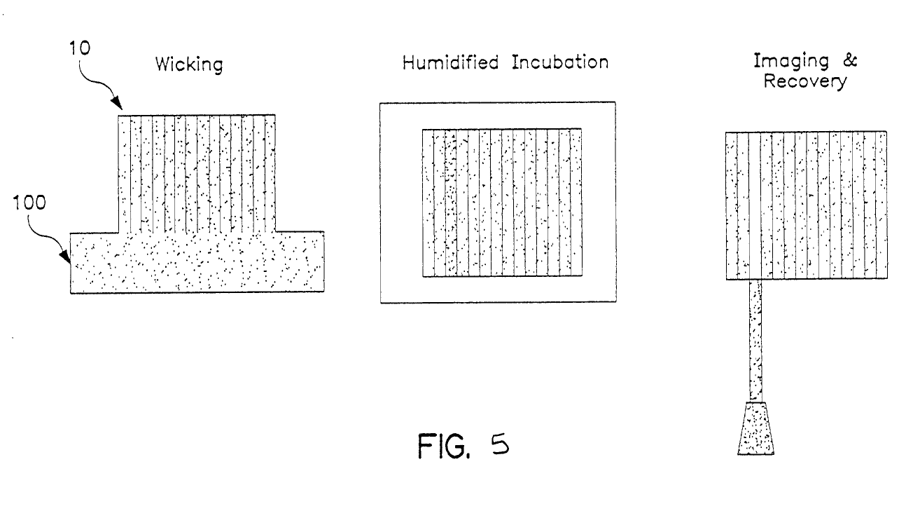

FIG. 5 depicts a general method of sample screening using a capillary array (10) according to the invention. In this depiction, capillary array (10) is immersed or contacted with a container (100) containing particles of interest. The particles can be cells, clones, molecules or compounds suspended in a liquid. The liquid is wicked into the capillary tubes by capillary action. The natural wicking that occurs as a result of capillary forces obviates the need for pumping equipment and liquid dispensers. A substrate for measuring biological activity (e.g., enzyme activity) can be contacted with the particles either before or after introduction of the particles into the capillaries in the capillary array. The substrate can include clones of a cell of interest, for example. The substrate can be introduced simultaneously into the capillaries by placing an open end of the capillaries in the container (100) containing a mixture of the particle-bearing liquid and the substrate. In some embodiments, it is a goal to achieve a certain concentration of particles of interest. A particular concentration of particles may also be achieved by dilution. FIGS. 8A-C show one such process, which is described below. Alternatively, the particle-bearing liquid may be wicked a portion of the way into the capillaries, and then the substrate is wicked into a remaining portion of the capillaries.

The mixture in the capillaries can then be incubated for producing a desired activity. The incubation can be for a specific period of time and at an appropriate temperature necessary for cell growth, for example, or to allow the substrate to permeabilize the cell membrane to produce an optically detectable signal, or for a period of time and at a temperature for optimum enzymatic activity. The incubation can be performed, for example, by placing the capillary array in a humidified incubator or in an apparatus containing a water source to ensure reduced evaporation within the capillary tubes. Evaporative loss may be reduced by increasing the relative humidity (e.g., by placing the capillary array in a humidified chamber). The evaporation rate can also be reduced by capping the capillaries with an oil, wax, membrane or the like. Alternatively, a high molecular weight fluid such as various alcohols, or molecules capable of forming a molecular monolayer, bilayers or other thin films (e.g., fatty acids), or various oils (e.g., mineral oil) can be used to reduce evaporation.

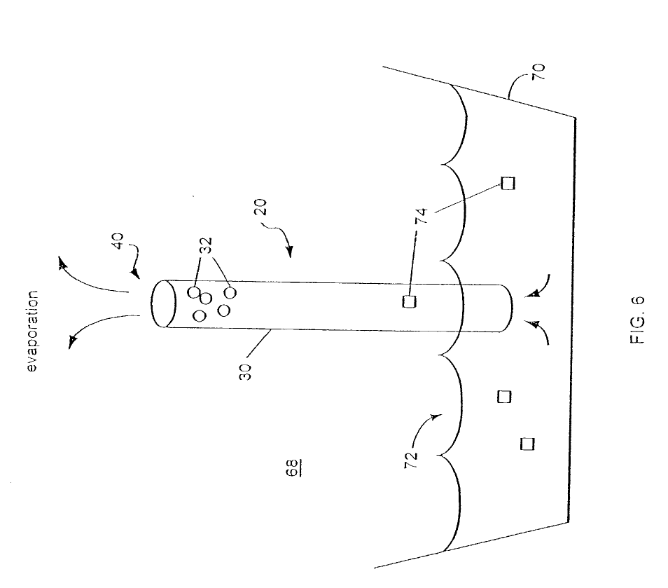

FIG. 6 illustrate a method for incubating a substrate solution containing cells of interest. While only a single capillary (20) is shown in FIG. 6 for simplicity, it should be understood that the incubation method applies to a capillary array having a plurality of capillaries (20). In accordance with one embodiment, a first fluid is wicked into the capillary (20) according to methods described above. The capillary (20) containing the substrate solution and cells (32) is then introduced to a fluid bath (70) containing a second liquid (72). The second liquid may or may not be the same as the first. For instance, the first liquid may contain particles (32) from which an activity is screened. The particles (32) are suspended in liquid within the lumen (40), and gradually migrate toward the top of the lumen (40) in the direction of the flow of liquid through the capillary (20) due to evaporation. The width of the lumen (40) at the open end of the capillary (20) is sized to provide a particular surface area of liquid at the top of the lumen (40), for controlling the amount and rate of evaporation of the liquid mixture. By controlling the environment (68) near the non-submersed end of the capillary (20), the first liquid from within the capillary (20) will evaporate, and will be replenished by the second liquid (72) from the fluid bath (70).

The amount of evaporation is balanced against possible diffusion of the contents of the capillary (20) into the liquid (72), and against possible mechanical mixing of the capillary contents with the liquid (72) due to vibration and pressure changes. The greater the width of the lumen (40), the larger the amount of mechanical mixing. Therefore, the temperature and humidity level in the surrounding environment may be adjusted to produce the desired evaporative cycle, and the lumen (40) width is sized to minimize mechanical mixing, in addition to produce a desired evaporation rate. The non-submersed open end of the capillary (20) may also be capped to create a vacuum force for holding the capillary contents within the capillary, and minimizing mechanical mixing and diffusion of the contents within the liquid (72). However when capped, the capillary (20) will not experience evaporation.

The liquid (72) can be supplemented with nutrients (74) to support a greater likelihood or rate of activity of the particles (32). For example, oxygen can be added to the liquid to nourish cells or to optimize the incubation environment of the cells. In another example, the liquid (72) can contain a substrate or a recombinant clone, or a developer for the particles (32). The cells can be optimally cultured by controlling the amount and rate of evaporation. For instance, by decreasing relative humidity of the environment (68), evaporation from the lumen (40) is increased, thereby increasing a rate of flow of liquid (72) through the capillary (20). Another advantage of this method is the ability to control conditions within the capillary (20) and the environment (68) that are not otherwise possible.

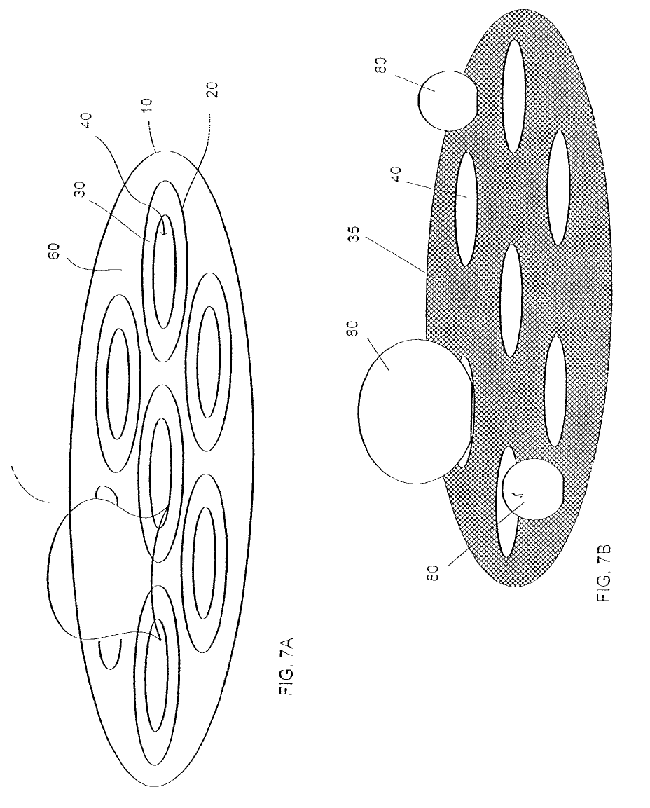

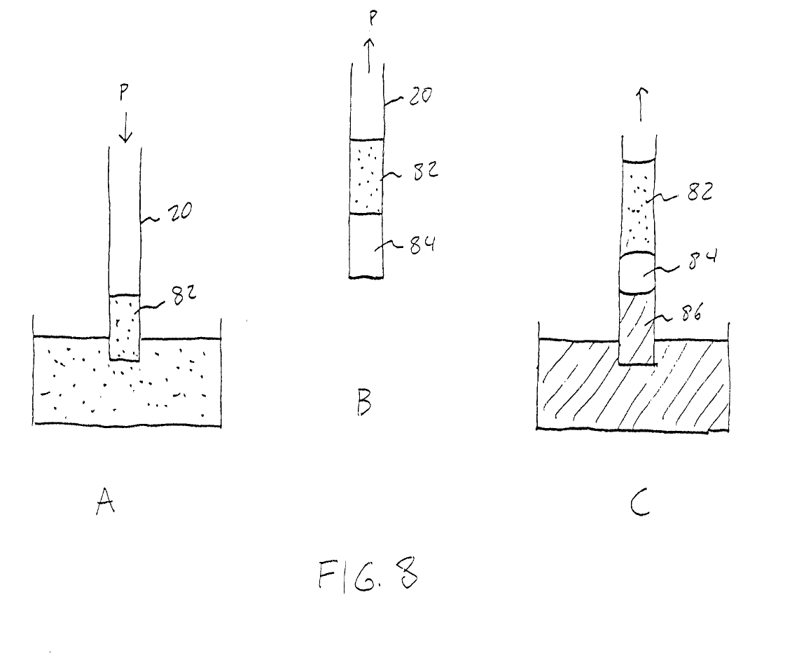

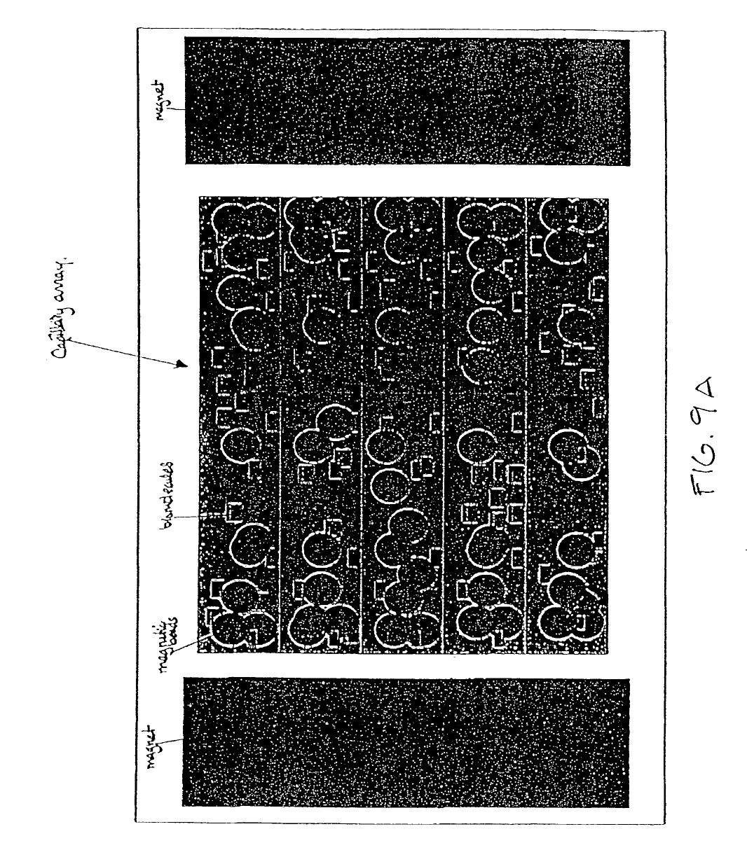

A relatively high humidity level of the environment will slow the rate of evaporation and keep more liquid within the capillary (20). If a temperature differential exists between a capillary array (10) and its environment, however, condensation can form on or near the ends of tightly-packed capillaries of the capillary array. FIG. 7A shows a portion of a capillary array (10) of the invention, to depict a situation in which a condensation bead (80) forms on the outer edge surface of several capillary walls (30), creating a potential conduit or bridge for “crosstalk” of matter between adjacent capillary tubes (20). The outer edge surface of the capillary walls (30) is preferably a planar surface. In an embodiment in which the wall (30) of the capillary (20) is glass, the outer edge surface of the capillary wall (30) can be polished glass.

In order to minimize the effects of such condensation, a hydrophobic coating (35) is provided over the outer edge surface of the capillary walls (30), as depicted in FIG. 7B. The coating (35) reduces the tendency for water or other liquid to accumulate near the outer edge surface of the capillary wall (30). Condensation will form either as smaller beads (82), be repelled from the surface of the capillary array, or form entirely over an opening to the lumen (40). In the latter case, the condensation bead (80) can form a cap to the capillary (20). In one embodiment, the hydrophobic coating (35) is TEFLON. In one configuration, the coating (35) covers only the outer edge surfaces of the capillary walls (30). In another configuration, the coating (35) can be formed over both the interstitial material (60) and the outer edge surfaces of the capillary walls (30). Another advantage of a hydrophobic coating (35) over the outer edge surface of the capillary tubes is during the initial wicking process, some fluidic material in the form of droplets will tend to stick to the surface in which the fluid is introduced. Therefore, the coating (35) minimizes extraneous fluid from forming on the surface of a capillary array (10), dispensing with a need to shake or knock the extraneous fluid from the surface.

In some instances, it is necessary to have more than one component in a capillary that are not premixed, and which can by later combined by dilution or mixing. FIGS. 8A-C show a dilution process that may be used to achieve a particular concentration of particles. In one embodiment employing dilution, a bolus of a first component (82) is wicked into a capillary (20) by capillary action until only a portion of the capillary (20) is filled. In one particular embodiment, pressure is applied at one end of the capillary (20) to prevent the first component from wicking into the entire capillary (20). The end (21) of the capillary may be completely or partially capped to provide the pressure.

An amount of air (84) is then introduced into the capillary adjacent the first component. The air (84) can be introduced by any number of processes. One such process includes moving the first component (82) in one direction within the capillary until a suitable amount of the air (84) is introduced behind the first component (82). Further movement of the first component (82) by a pulling and/or pushing pressure causes a piston-like action by the first component (82) on the air.

The capillary (20) or capillary array is then contacted to a second component (86). The second component (86) is preferably pulled into the capillary (20) by the piston-like action created by movement of the first component (82), until a suitable amount of the second component (86) is provided in the capillary, separated from the first component by the air (84). One of the first or second components may contain one or more particles of interest, and the other of the components may be a developer of the particles for causing an activity of interest. The capillary or capillary array can then be incubated for a period of time to allow the first and second components to reach an optimal temperature, or for a sufficient time to allow cell growth for example. The air-bubble separating the two components can be disrupted in order to allow mix the two components together and initialize the desired activity. Pressure can be applied to collapse the bubble. In one example, the mixture of the first and second components starts an enzymatic activity to achieve a multi-component assay.

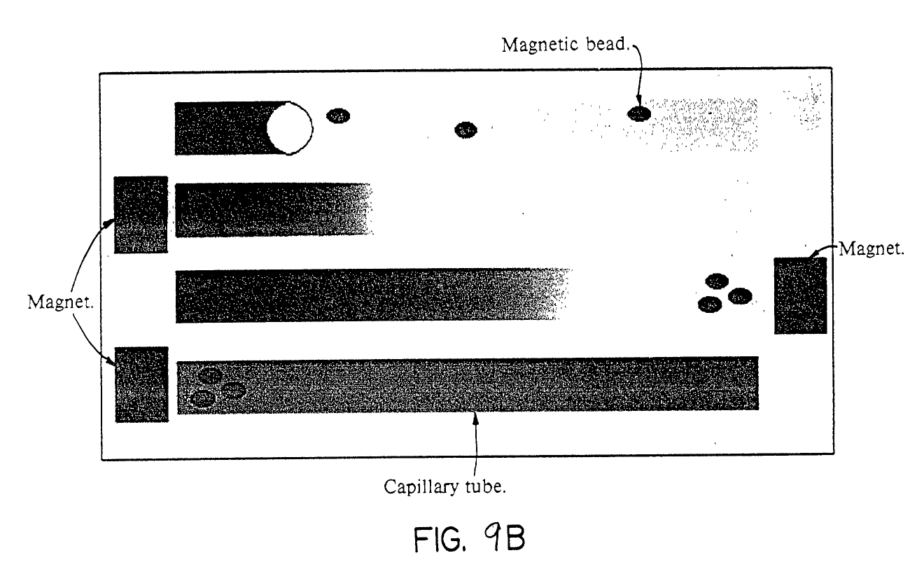

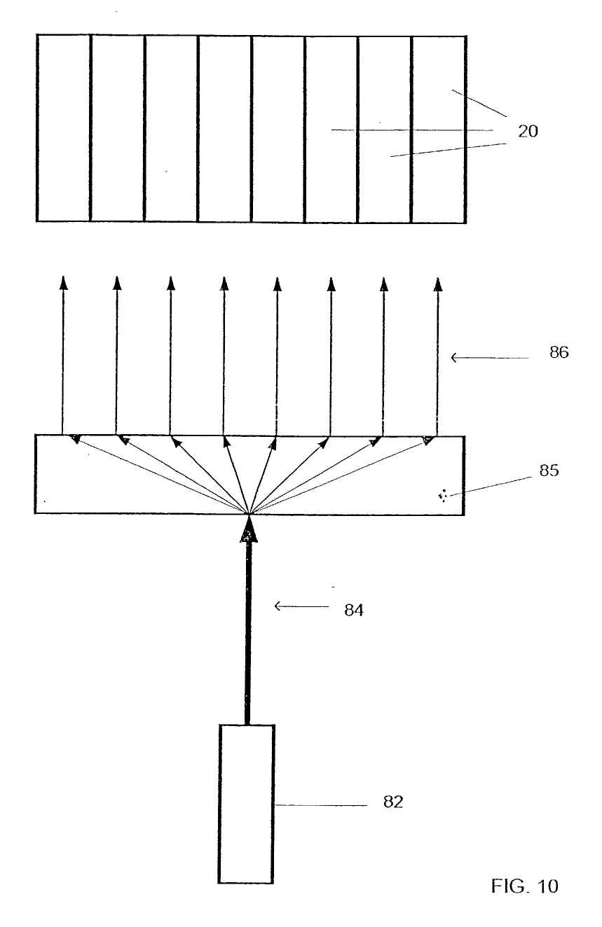

Paramagnetic beads contained within a capillary (20) can be used to disrupt the air bubble and/or mix the contents of the capillary (20) or capillary array (10). For example, FIGS. 9A and 9B depict an embodiment of the invention in which paramagnetic beads are magnetically moved from one location to another location. The paramagnetic beads are attracted by magnetic fields applied in proximity to the capillary or capillary array. By alternating or adjusting the location of the magnetic field with respect to each capillary, the paramagnetic beads will move within each capillary to mix the liquid therein. Mixing the liquid can improve cell growth by increasing aeration of the cells. The method also improves consistency and detectability of the liquid sample among the capillaries.

In another embodiment, a method of forming a multi-component assay includes providing one or more capsules of a second component within a first component. The second component capsules can have an outer layer of a substance that melts or dissolves at a predetermined temperature, thereby releasing the second component into the first component and combining particles among the components. A thermally activated enzyme may be used to dissolve the outer layer substance. Alternatively, a “release on command” mechanism that is configured to release the second component upon a predetermined event or condition may also be used.

In another embodiment, recombinant clones containing a reporter construct or a substrate are wicked into the capillary tubes of the capillary array. In this embodiment, it is not necessary to add a substrate as the reporter construct or substrate contained in the clone can be readily detected using techniques known in the art. For example, a clone containing a reporter construct such as green fluorescent protein can be detected by exposing the clone or substrate within the clone to a wavelength of light that induces fluorescence. Such reporter constructs can be implemented to respond to various culture conditions or upon exposure to various physical stimuli (including light and heat). In addition, various compounds can be screened in a sample using similar techniques. For example, a compound detectably labeled with a florescent molecule can be readily detected within a capillary tube of a capillary array.

In yet another embodiment, instead of dilution, a fluorescence-activated cell sorter (FACS) is used to separate and isolate clones for delivery into the capillary array. In accordance with this embodiment, one or more clones per capillary tube can be precisely achieved. In yet another embodiment, cells within a capillary are subjected to a lysis process. A chemical is introduced within one of the components to cause a lysis process where the cells burst.

Some assays may require an exchange of media within the capillary. In a media exchange process, a first liquid containing the particles is wicked into a capillary. The first liquid is removed, and replaced with a second liquid while the particles remain suspended within the capillary. Addition of the second liquid to the capillary and contact with the particles can initialize an activity, such as an assay, for example. The media exchange process may include a mechanism by which the particles in the capillary are physically maintained in the capillary while the first liquid is removed. In one embodiment, the inner walls of the capillary array are coated with antibodies to which cells bind. Then, the first liquid is removed, while the cells remain bound to the antibodies, and the second liquid is wicked into the capillary. The second liquid could be adapted to cause the cells to unbind if desirable. In an alternative embodiment, one or more walls of the capillary can be magnetized. The particles are also magnetized and attracted to the walls. In still another embodiment, magnetized particles are attracted and held against one side of the capillary upon application of a magnetic field near that side.

The capillary array is analyzed for identification of capillaries having a detectable signal, such as an optical signal (e.g., fluorescence), by a detector capable of detecting a change in light production or light transmission, for example. Detection may be performed using an illumination source that provides fluorescence excitation to each of the capillaries in the array, and a photodetector that detects resulting emission from the fluorescence excitation. Suitable illumination sources include, without limitation, a laser, incandescent bulb, light emitting diode (LED), arc discharge, or photomultiplier tube. Suitable photodetectors include, without limitation, a photodiode array, a charge-coupled device (CCD), or charge injection device (CID).

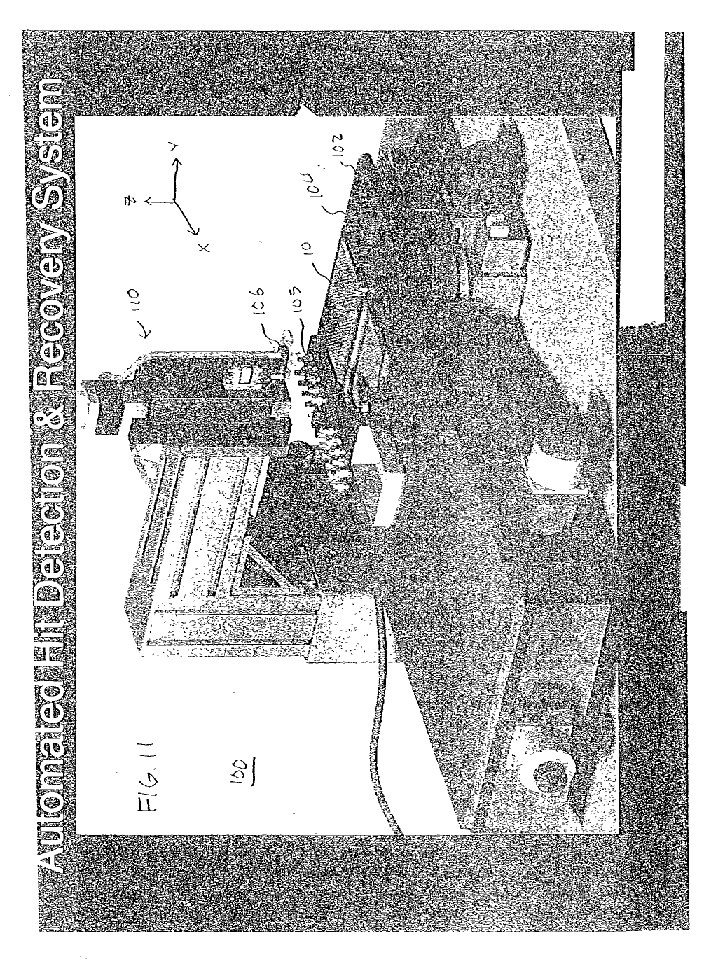

In one embodiment, shown with reference to FIG. 10, a detection system includes a laser source (82) that produces a laser beam (84). The laser beam (84) is directed into a beam expander (85) configured to produce a wider or less divergent beam (86) for exciting the array of capillaries (20). Suitable laser sources include argon or ion lasers. For this embodiment, a cooled CCD can be used.

The light generated by, for example, enzymatic activation of a fluorescent substrate is detected by an appropriate light detector or detectors positioned adjacent to the apparatus of the invention. The light detector may be, for example, film, a photomultiplier tube, photodiode, avalanche photo diode, CCD or other light detector or camera. The light detector may be a single detector to detect sequential emissions, such as a scanning laser. Or, the light detector may include a plurality of separate detectors to detect and spatially resolve simultaneous emissions at single or multiple wavelengths of emitted light. The light emitted and detected may be visible light or may be emitted as non-visible radiation such as infrared or ultraviolet radiation. A thermal detector may be used to detect an infrared emission. The detector or detectors may be stationary or movable.

Illumination can be channeled to particles of interest within the array by means of lenses, mirrors and fiber optic light guides or light conduits (single, multiple, fixed, or moveable) positioned on or adjacent to at least one surface of the capillary array. A detectable signal, such as emitted light or other radiation, may also be channeled to the detector or detectors by the use of such mechanisms.

The photodetector preferably comprises a CCD, CID or an array of photodiode elements. Detection of a position of one or more capillaries having an optical signal can then be determined from the optical input from each element. Alternatively, the array may be scanned by a scanning confocal or phase-contrast fluorescence microscope or the like, where the array is, for example, carried on a movable stage for movement in a X-Y plane as the capillaries in the array are successively aligned with the beam to determine the capillary array positions at which an optical signal is detected. A CCD camera or the like can be used in conjunction with the microscope. The detection system is preferably computer-automated for rapid screening and recovery. In a preferred embodiment, the system uses a telecentric lens for detection. The magnification of the lens can be adjusted to focus on a subset of capillaries in the capillary array. At one extreme, for instance, the detection system can have a 1:1 correlation of pixels to capillaries. Upon detecting a signal, the focus can be adjusted to determine other properties of the signal. Having more pixels per capillary allows for subsequent image processing of the signal.

Where a chromogenic substrate is used, the change in the absorbance spectrum can be measured, such as by using a spectrophotometer or the like. Such measurements are usually difficult when dealing with a low-volume liquid because the optical path length is short. However, the capillary approach of the present invention permits small volumes of liquid to have long optical path lengths (e.g., longitudinally along the capillary tube), thereby providing the ability to measure absorbance changes using conventional techniques.

A fluid within a capillary will usually form a meniscus at each end. Any light entering the capillary will be deflected toward the wall, except for paraxial rays, which enter the meniscus curvature at its center. The paraxial rays create a small bright spot in middle of capillary, representing the small amount of light that makes it through. Measurement of the bright spot provides an opportunity to measure how much light is being absorbed on its way through. In a preferred embodiment, a detection system includes the use of two different wavelengths. A ratio between a first and a second wavelength indicates how much light is absorbed in the capillary. Alternatively, two images of the capillary can be taken, and a difference between them can be used to ascertain a differential absorbance of a chemical within the capillary.

In absorbance detection, only light in the center of the lumen can travel through the capillary. However, if at least one meniscus is flattened, the optical efficiency is improved. The meniscus can be kept flat under a number of circumstances, such as during a continuous cycle of evaporation, discussed above with reference to FIG. 6. In that embodiment, the fluid bath can be contained in a clear, light-passing container, and the light source can be directed through the fluid bath into the capillary.

In another embodiment, bioactivity or a biomolecule or compound is detected by using various electromagnetic detection devices, including, for example, optical, magnetic and thermal detection. In yet another embodiment, radioactivity can be detected within a capillary tube using detection methods known in the art. The radiation can be detected at either end of the capillary tube.

Other detection modes include, without limitation, luminescence, fluorescence polarization, time-resolved fluorescence. Luminescence detection includes detecting emitted light that is produced by a chemical or physiological process associated with a sample molecule or cell. Fluorescence polarization detection includes excitation of the contents of the lumen with polarized light. Under such environment, a fluorophore emits polarized light for a particular molecule. However, the emitting molecule can be moving and changing its angle of orientation, and the polarized light emission could become random.

Time-resolved fluorescence includes reading the fluorescence at a predetermined time after excitation. For a relatively long-life fluorophore, the molecule is flashed with excitation energy, which produces emissions from the fluorophore as well as from other particles within the substrate. Emissions from the other particles causes background fluorescence. The background fluorescence normally has a short lifetime relative to the long-life emission from the fluorophore. The emission is read after excitation is complete, at a time when all background fluorescence usually has short lifetime, and during a time in which the long-life fluorophores continues to fluoresce. Time-resolved fluorescence are therefore a technique for suppressing background fluorescent activity.

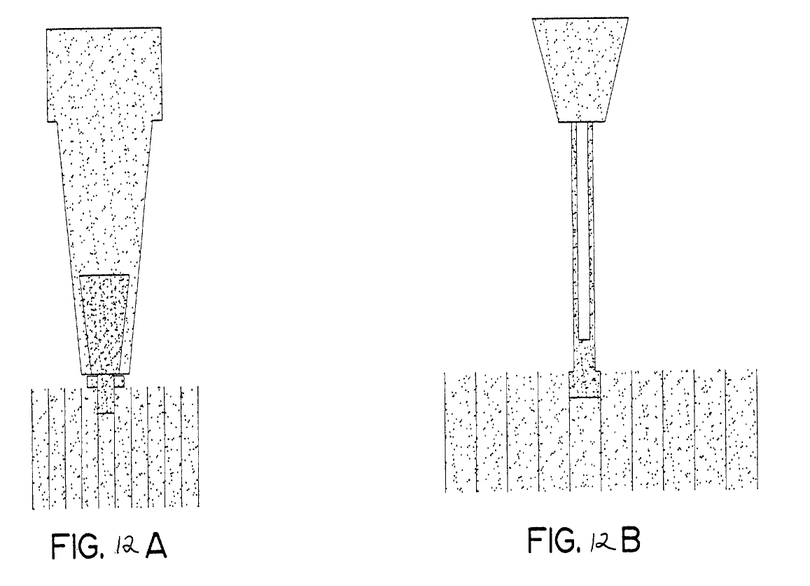

Recovery of putative hits (cells or clones producing a detectable or optical signal) can be facilitated by using position feedback from the detection system to automate positioning of a recovery device (e.g., a needle pipette tip or capillary tube). FIG. 11 shows an example of a recovery system (100) of the invention. In this example, a needle 105 is selected and connected to recovery mechanism (106). A support table (102) supports a capillary array (10) and a light source (104). The light source is used with a camera assembly (110) to find an X, Y and Z coordinate location of a needle (105) connected to the recovery mechanism (106). The support table is moved relative to the capillary array in the X and Y axes, in order to place the capillary array (10) underneath the needle (105), where the capillary array (10) contains a “hit.” According to various embodiments, each section of a recovery system can be moved or kept stationary.

The recovery mechanism (106) then provides a needle (105) to a capillary containing a “hit” by overlapping the tip of the needle (105) with the capillary containing the “hit,” in the Z direction, until the tip of the needle engages the capillary opening. In order to avoid damage to the capillary itself the needle may be attached to a spring or be of a material that flexes. Once in contact with the opening of the capillary the sample can be aspirated or expelled from the capillary. Alternatively, the capillary array may be moved relative to a stationary needle (105), or both moved.

In a specific exemplary embodiment of a recovery technique, a single camera is used for determining a location of a recovery tool, such as the tip of a needle, in the Z-plane. The Z-plane determination can be accomplished using an auto-focus algorithm, or proximity sensor used in conjunction with the camera. Once the proximity of the recovery tool in Z is known, an image processing function can be executed to determine a precise location of the recovery tool in X and Y. In one embodiment, the recovery tool is back-lit to aid the image processing. Once the X and Y coordinate locations are known, the capillary array can be moved in X and Y relative to the precise location of the recovery tool, which can be moved along the Z axis for coupling with a target capillary.

In an alternative specific embodiment of a recovery technique, two or more cameras are used for determining a location of the recovery tool. For instance, a first camera can determine X and Z coordinate locations of the recovery tool, such as the X, Z location of a needle tip. A second camera can determine Y and Z coordinate locations of the recovery tool. The two sets of coordinates can then be multiplexed for a complete X,Y,Z coordinate location. Next, the movement of the capillary array relative to the recovery tool can be executed substantially as above.

The sample can be expelled by, for example, injecting a blast of inert gas or fluid into the capillary and collecting the ejected sample in a collection device at the opposite end of the capillary. The diameter of the collection device can be larger than or equal to the diameter of the capillary. The collected sample can then be further processed by, for example, extracting polynucleotides, proteins or by growing the clone in culture.

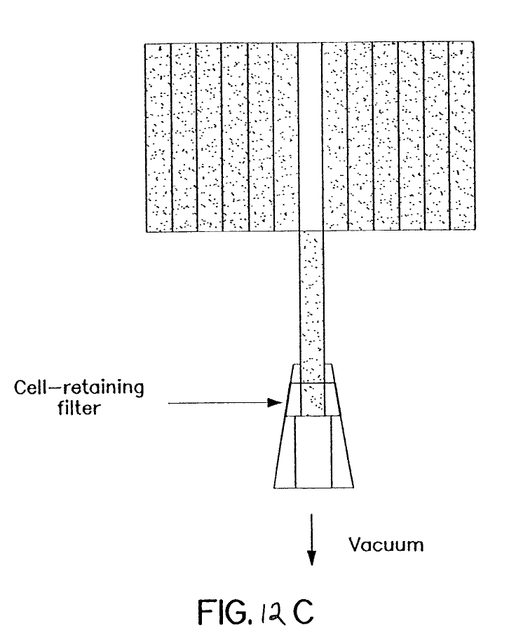

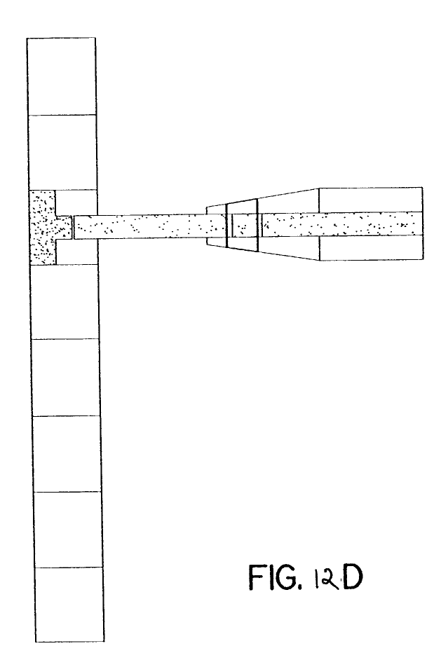

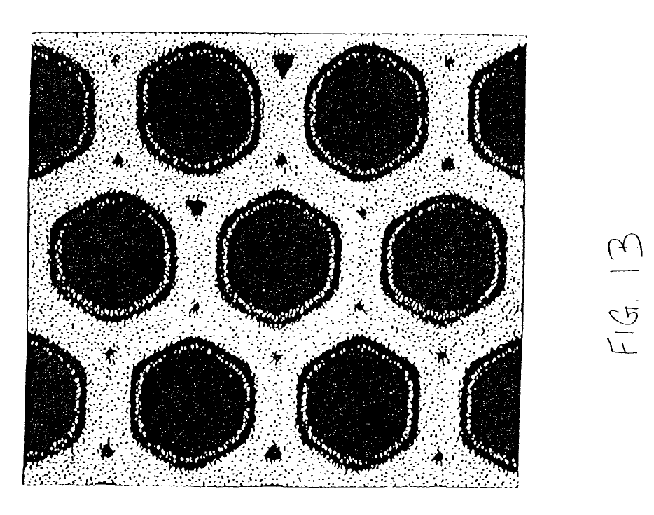

In another embodiment, the sample is aspirated by use of a vacuum. In this embodiment, the needle contacts, or nearly contacts, the capillary opening and the sample is “vacuumed” or aspirated from the capillary tube onto or into a collection device. The collection device may be a microfuge tube or a filter located proximal to the opening of the needle, as depicted in FIGS. 12A-D. FIG. 12D shows further processing of a sample collected onto a filter following aspiration of the sample from the capillary. The sample includes particles, such as cells, proteins, or nucleic acids, which when present on the filter, can be delivered into a collection device. Suitable collection devices include a microfuge tube, a capillary tube, microtiter plate, cell culture plate, and the like. The delivery of the sample can be accomplished by forcing another media, air or other fluid through the filter in the reverse direction.

The sample can also be expelled from a capillary by a sample ejector. In one embodiment, the ejector is a jet system where sample fluid at one end of the capillary tube is subjected to a high temperature, causing fluid at the other end of the capillary tube to eject out. The heating of fluid can be accomplished mechanically, by applying a heated probe directly into one end of a capillary tube. The heated probe preferably seals the one end, heats fluid in contact with the probe, and expels fluid out the other end of the capillary tube. The heating and expulsion may also be accomplished electronically. For instance, in an embodiment of the jet system, at least one wall of a capillary tube is metalized. A heating element is placed in direct contact with one end of the wall. The heating element may completely close off the one end, or partially close the one end. The heating element charges up the metalized wall, which generates heat within the fluid. The heating element can be an electricity source, such as a voltage source, or a current source. In still yet another embodiment of a jet system, a laser applies heat pulses to the fluid at one end of the capillary tube.

Other systems for expelling fluid from a capillary tube of the invention are possible. An electric field may be created in or near the fluid to create an electrophoretic reaction, which causes the fluid to move according to electromotive force created by the electric field. A electromagnetic field may also be used. In one embodiment, one or more capillaries contain, in addition to the fluid, magnetically charged particles to help move the fluid or magnetized particles out of the capillary array.

While the invention has been described in detail with reference to certain preferred embodiments thereof, it will be understood that modifications and variations are within the spirit and scope of that which is described and claimed.

What is claimed is:

1. A capillary for screening a sample, wherein the capillary is adapted for being held in an array of capillaries, the capillary comprising:
a first wall defining a lumen for retaining the sample, wherein the first wall forms a waveguide for propagating detectable signals therein; and
a second wall formed of a filtering material, for filtering excitation energy provided to the lumen to excite the sample.

2. The capillary of claim 1, wherein the second wall circumscribes the first wall.

3. The capillary of claim 1, wherein the second wall is formed of extra mural absorption (EMA) glass.

4. The capillary of claim 3, wherein the EMA glass is tuned to filter specific wavelengths of light.

5. A capillary array for screening a plurality of samples, comprising:
a plurality of capillaries, held together into the array, wherein each capillary includes a first wall defining a lumen for retaining the sample, and a second wall circumscribing the first wall, for filtering excitation energy provided to the lumen to excite the sample.

6. The array of claim 5, wherein the second wall of each capillary is formed of a filtering material.

7. The array of claim 6, wherein the filtering material is EMA glass.

8. The array of claim 7, wherein the EMA glass is tuned to filter specific wavelengths of light.

9. The array of claim 5, further comprising interstitial material between adjacent capillaries.

10. The array of claim 9, wherein the interstitial material is adapted to absorb light.
